(19) United States
(12) Patent Application Publication
(10) Pub. No.: US 2023/0326607 A1
(43) Pub. Date: Oct. 12, 2023

(54) SYSTEM FOR AND METHOD OF DETERMINING, BASED ON INPUT ASSOCIATED WITH A PERSON, A HEALTH STATUS SCORE

(71) Applicant: Nederlandse Organisatie voor toegepast-natuurwetenschappelijk onderzoek TNO, 's-Gravenhage (NL)

(72) Inventors: Timothy Jerzy VAN DEN BROEK, Utrecht (NL); Suzan WOPEREIS, Woerden (NL)

(21) Appl. No.: 18/132,568

(22) Filed: Apr. 10, 2023

Related U.S. Application Data
(63) Continuation of application No. PCT/NL2022/050201, filed on Apr. 12, 2022.

(51) Int. Cl. G16H 50/30; G16H 10/60; G16H 50/20

(52) U.S. Cl. CPC G16H 50/30; G16H 10/60; G16H 50/20

(57) ABSTRACT

A system is described that facilitates determining, based on input associated with a person, a health status score associated with the person. The input relates to parameter values of at least one parameter relating to traits of the person. The system receives the input, and a processor executes a first machine learning data processing model for generating, based on the input data, a plurality of candidate records. For each candidate record, a parameter value combination formed by entered parameter values and candidate parameter values forms a unique combination. The data processing model generates, for each candidate record, a likelihood value indicative of a probability that the parameter value combination of the candidate record provides a true representation of the traits of the person.

### CROSS-REFERENCE TO RELATED APPLICATIONS

This application is a continuation of PCT International Application No. PCT/NL2022/050201, filed Apr. 12, 2022, the contents of which are hereby incorporated by reference in their entirety.

### FIELD OF THE INVENTION

The present invention is directed at a system for determining, based on input associated with a person, a health status score associated with the person, and wherein the input relates to parameter values of one or more parameters from a parameter set, the parameter set comprising a plurality of defined parameters, the parameters relating to traits of the person, wherein the system comprises: an input means for receiving the input, wherein the input comprises input data representing entered parameter values of at least three parameters from the plurality of parameters of the parameter set; wherein the system further comprises a processor configured for executing a first machine learning data processing model.

### BACKGROUND

Stimulated by the increased attention and public focus on personal health, in the past decennia various personal health monitoring solutions have been developed. The ubiquitous availability of personal computing devices, such as smart watches, smart phones, tablet computers, laptops and the vast amount of all other types of smart devices, brings the availability of these systems to the individual user in his/her personal environment, enabling to perform personal health checks without the aid of a health professional or medical specialist.

To be able to provide an accurate prediction, health monitoring methods and systems require to receive input data relating to many different health related parameters, i.e. with respect to different traits (e.g. habits, body conditions, body properties, health history, current diseases or disorders) of a person. Because this data is in many cases not readily available or not available at all to a user and therefore calculating health scores is for the above reason often based on incomplete data, a suboptimal health score result may at best be achievable by such systems.

### SUMMARY OF THE INVENTION

The present document is directed at providing a method and system that overcome this shortcoming, and which enable to provide or at least converge towards a more accurate prediction of a health status.

To this end, in accordance with a first aspect, there is provided herewith a system for determining, based on input associated with a person, a health status score associated with the person. The input relates to parameter values of one or more parameters from a parameter set, wherein the parameter set comprises a plurality of defined parameters. These parameters relate to traits of the person. The system comprises an input means for receiving the input, wherein the input comprises input data representing entered parameter values of at least three parameters from the plurality of parameters of the parameter set. The system further comprises a processor configured for executing a first machine learning data processing model.

The first machine learning data processing model is configured for generating, based on the input data, a plurality of candidate records. Each candidate record comprises: the entered parameter values of the at least three parameters of the parameter set, and for each further parameter of the parameter set different from the at least three parameters, a candidate parameter value. The candidate parameter value thereby is generated by the first machine learning data processing model. The candidate records are generated such that, for each candidate record, a parameter value combination formed by the entered parameter values and the candidate parameter values forms a unique combination within the plurality of parameter value combinations of the candidate records. The first machine learning data processing model is further configured for generating, for each candidate record, a likelihood value indicative of a probability that the parameter value combination of the candidate record provides a true representation of the traits of the person. Furthermore, the first machine learning data processing model is configured for generating said candidate value for each further parameter, based on the entered parameter values.

In accordance with the present invention, the first machine learning data processing model enables to complete the input data by generating a plurality of candidate records. The candidate records form virtual twins of the person in question, in the sense that each of these records includes the entered parameter values of the at least three parameters received as input data via the input means. Each record is enriched with generated parameter values: the candidate parameter values. These candidate parameter values are generated by the first machine learning data processing model, which is trained to generate for each different parameter of the parameter set (i.e. different from the at least three parameters), a plurality of different candidate parameter values. The first machine learning data processing model uses the entered parameter values as set values, which are thereby considered as reliable and accurate data. With each candidate record, which thereby provides a potential representation of the person in question in terms of all the parameters of the parameter set, the first machine learning data processing model generates a likelihood value which is indicative of a probability that the parameter value combination of the candidate record provides a true representation of the traits of the person. Therefore, overall, the set of candidate records obtained in this manner provides a population of virtual non-existing persons that have the entered parameter values for the at least three parameters in common, but for which the other parameters vary from virtual person to virtual person. The likelihood value associated with each of these virtual persons, indicates the probability that remaining parameters of the actual person in consideration indeed matches the generated candidate parameter values of this virtual person.

The population of candidate records with their likelihood values obtained in this manner, may thereafter be used to perform statistical analysis in order to provide an estimated health score. In some embodiments, an estimate of the accuracy or reliability of the health status score is calculated, expressed as an error value to be associated with this estimated health score. This error value may be provided as output to a user together with the health status score, such that the user is made aware of the error value. An advantage thereof is that the user (which may be the person in consideration by the system) is stimulated to obtain real acquired values of some parameters, and to include these values as input data in the input. The number of entered values for parameters from the parameter set is increased thereby, such that the accuracy health status score increases and the error value decreases. As may be appreciated, the more parameter values that are entered, the more accurate the candidate records become in truly representing the traits of the person in question. If all parameters would be entered, all parameter values are exactly known (as received via input) and no candidate parameter values need to be generated.

The first machine learning data processing model may for example be a probabilistic statistical model, such as a Bayesian Network model. A Bayesian network is a probabilistic graphical model that represents a set of variables and their conditional dependencies via a directed acyclic graph (DAG). Bayesian networks are known for considering an occurred event and predicting the likelihood that any one of several possible known causes was a contributing factor. However, underlying the present invention is the realization that a Bayesian network may also be applied for compensating for missing input data, i.e. by using the probabilistic statistical model to estimate likely parameter values that go along with the entered parameter values. These entered parameter values, i.e. the at least three parameters at input, preferably in accordance with an embodiment comprised by age, gender and ethnicity. Based on these three parameters as elementary parameters, a large number of other parameters may be generated to go along therewith, and likelihood values for such generated candidate parameter values can be determined using the statistical model. The invention is not limited to the application of a Bayesian network model as the first machine learning data processing model. Alternatively, other machine learning data processing models may be applied in order to compensate for missing input data. To provide some examples of alternatives, the first machine learning data processing model may likewise comprise or be formed by: a variational autoencoder (VAE) or a generative adversarial network (GAN).

In some embodiments, the processor is further configured for determining, using the parameter value combinations of the candidate records and the likelihood values associated with the candidate records, the health status score of said person associated with the input, wherein the health status score is based on the candidate records. The health status score may for example be obtained based on a statistical algorithm or by assuming that a most likely one of the candidate record as representative for the person in consideration. In some of these embodiments, for example, for determining the health status score the processor is further configured for executing a second machine learning data processing model, wherein the second machine learning data processing model is configured for determining, for each candidate record, an individual health status score associated with the candidate record; and wherein, for determining the health status score of said person, the processor is further configured for calculating a weighed mean of the individual health status scores weighed based on the associated likelihood values of each candidate record. The application of the second machine learning data processing model enables to efficiently converge the plurality of candidate records and likelihood values into a final health status score. For example, the second machine learning data processing model may be a principal component analysis model, although alternatively other types of machine learning models may be applied in order to process the different candidate records and determine the health status score. Alternative models that may be applied as second machine learning data processing model may include an independent component analysis model (ICA), a multidimensional scaling model (MDS), a singular value decomposition (SVD), or a non-negative matrix factorization (NMF), each providing similar or acceptable results.

In an embodiment, a national health database is used for training the first, the second, or both machine learning data processing models. The national health database may be database that consists of population health statistics data of a European or Asian population.

In another embodiment, the NHANES database is used for training the first and second machine learning data processing models, specifically a database related to the areas of metabolic health, cardiovascular health, muscle health, immune health, and weight management. The NHANES database is a national health database that consists of population health statistics data of the United States population. The data is obtained by the National Health and Nutrition Examination Survey conducted by the National Center for Health Statistics (NCHS).

In other or further embodiments, the processor is further configured for scaling the health status score by multiplying the health status score with a scaling factor, wherein the scaling factor is dependent on the at least three parameters. The scaling enables to mitigate the effects of extreme situations. In particular, when dealing with human individuals that can experience stress or discouragement, a built-in scaling factor is an effective manner of making an end result more palatable and useful. For example, suppose that the health status score would be expressed or provided in the form of a biological age as compared to the actual physical age, then if the in consideration person is a 25 year old man (physical age) and the calculated health status score would indicate a biological age of 16 years old, the person in question may become disappointed or dissatisfied with this. In that case, by mitigating the resulting health status score for example such that it indicates a biological age of 21 years old, the score becomes more acceptable to the person in consideration whereas the message conveyed is the same: “your body is relatively young for your age”. In order to implement such a scaling, in some embodiments, the processor is configured for obtaining an algorithm for determining the scaling factor, wherein for obtaining the algorithm the processor is configured for: identifying a plurality of distinguished conditions, wherein each condition is represented by a unique combination of parameter values of the at least three parameters; applying the first machine learning data processing model for generating, for each condition and based on the unique combination associated with said condition, a plurality of model candidate records; calculating, for each condition, a modelled health status score associated with said condition; and performing a linear regression model for obtaining the algorithm. As may be appreciated, other ways to perform scaling may be applied, e.g. the most straightforward one being e.g. a limit to the score obtained.

The example of a biological age corresponds to an embodiment of the present invention, and the health status score may likewise include a different type of score. The term ‘physical age’ thereby indicates the real actual age of the person (i.e. the amount of time expired since the person's birth). The term ‘biological age’ in this context indicates a determined age on the basis of the actual health state of the person in consideration as compared to the mean health state of other persons having the same physical age, the latter being determinable based on statistics.

In accordance with a second aspect, the invention is directed at a method of determining, based on input associated with a person, a health status score associated with the person, and wherein the input relates to parameter values of one or more parameters from a parameter set, the parameter set comprising a plurality of defined parameters, the parameters relating to traits of the person, wherein the method comprises: receiving, by an input means, the input, wherein the input comprises input data representing entered parameter values of at least three parameters from the plurality of parameters of the parameter set; generating, by a first machine learning data processing model executed by a processor, a plurality of candidate records based on the input data, wherein each candidate record comprises: the entered parameter values of the at least three parameters of the parameter set; and for each further parameter of the parameter set different from the at least three parameters, a candidate parameter value; and wherein said generating is performed such that, for each candidate record, a parameter value combination formed by the entered parameter values and the candidate parameter values forms a unique combination within the plurality of parameter value combinations of the candidate records; the method further comprising: generating for each candidate record, by the first machine learning data processing model, a likelihood value indicative of a probability that the parameter value combination of the candidate record provides a true representation of the traits of the person; and wherein said generating, for each candidate record, of the candidate value for each further parameter is based on the entered parameter values.

Furthermore, in accordance with a third aspect, the invention relates to a training method for training of a first machine learning data processing method, prior to a determining of a health status score, wherein the training includes: obtaining, from a database, health statistics data, wherein the health statistics data comprises health parameter statistics for a population of persons; performing, based on the health statistics data, an iterative optimization algorithm such as to identify one or more conditional dependencies between a plurality of health parameters comprised by the health statistics data, wherein the one or more conditional dependencies quantify whether and to which degree any health parameter of the plurality of health parameters is dependent on any other health parameter of the plurality of health parameters; and terminating the iterative optimization algorithm upon identifying a stable set of conditional dependencies, wherein the set is determined as stable if upon any further iteration a change in any of the conditional dependencies is smaller than a predetermined threshold. The training method may be part of the method of the second aspect described above, or may be an independent method in order to provide a first machine learning data processing model to be used in a system or method of the invention.

In some embodiments, the method further comprises the steps of: obtaining a training data representing training parameter values of the at least three parameters from the plurality of parameters of the parameter set; generating, by the first machine learning data processing model, for each further parameter of the parameter set different from the at least three parameters, a generated parameter value; generating, by the first machine learning data processing model, a likelihood value indicative of a probability that a training combination of the training parameter values and the generated parameter values provides a true representation of the traits of the person; comparing the likelihood value with the health parameter statistics for verifying a correctness of the likelihood value; and modifying, dependent on the step of comparing, at least one of the one or more conditional dependencies and perform the iterative optimization algorithm. In some embodiments, the iterative optimization algorithm is a tabu search algorithm.

### BRIEF DESCRIPTION OF THE DRAWINGS

The invention will further be elucidated by description of some specific embodiments thereof, making reference to the attached drawings. The detailed description provides examples of possible implementations of the invention, but is not to be regarded as describing the only embodiments falling under the scope. The scope of the invention is defined in the claims, and the description is to be regarded as illustrative without being restrictive on the invention. In the drawings:

FIG. 1 schematically illustrates a method in accordance with an embodiment of the present invention;

FIG. 2 schematically illustrates a determination process of an overall health score, in accordance with an embodiment of the invention;

FIG. 3 schematically illustrates how a scaling algorithm may be determined using a simulated dataset, in an embodiment of the invention;

FIG. 4 provides an overview of an embodiment for calculating an overall health score 40, in accordance with the invention; and

FIG. 5 schematically illustrates a system 1 for determining a health status score in accordance with an embodiment.

### DETAILED DESCRIPTION

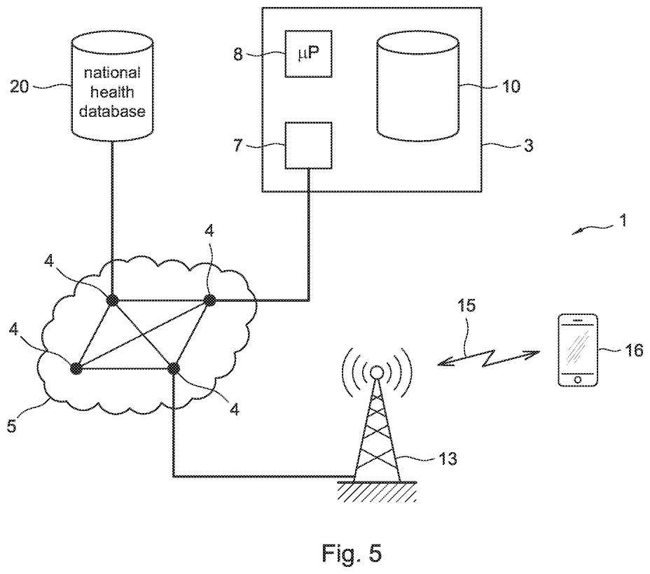

FIG. 5 schematically illustrates a system 1 for determining a health status score. The system 1 illustrated in FIG. 5 is merely a schematic illustration of an exemplary implementation of such a system, which is not intended to be restrictive on the scope of the invention in any way. The system 1 may be implemented in a different manner, as will be appreciated by the skilled person.

The system 1 of FIG. 5 may comprise a server 3 that is attached to a wide area network 5. The network 5 consists of a system of interconnected network nodes 4 that enables to transmit data over large distances to other interconnected network entities. The server 3, in the example illustrated, includes a communication unit 7 and a processor 8. Furthermore the server 3 includes an internal memory 10 for storing data. The memory 10 may comprise one or more machine learning data processing models, such as a Bayesian network model and a principal component analysis model as will be described further below. However, any of these machine learning data processing models may likewise be stored on an external server or database unit that may be accessed via the network 5.

The network 5 further connects with a base station 13 of a mobile telecommunications network. Through the base station 13, mobile telephone 16 of a user transmits data via a wireless connection 15 to the telecommunication network 5. This data is received as input data by server 3 through communication unit 7. Furthermore, the network 5 also provides access to a national health database 20. The national health database 20 comprises population health statistics data for example of the population of a country. This health statistics data, as will be described further below, will be used to train machine learning data processing models (e.g. models 32 and 55) and to perform various types of statistical analysis to the benefit of the system 1 of the present invention.

The input data to be provided for example via mobile telecommunication unit 16 through the network 5 to the server 3 may consist of entered parameter values that are provided by the user of mobile telephone 16 to the server 3. Although in the example of FIG. 5, the input data is provided via a mobile telephone, the skilled person may appreciate that many other kinds of communication means may be used for providing input to the server 3. For example, the user may use a laptop, a smart watch, an interconnected or smart medical device such as a blood pressure sensor or thermometer or a personal data file stored on a data repository to which the user provides access. The input data is received by the server 3, which uses the input data in order to perform a method in accordance with the present invention.

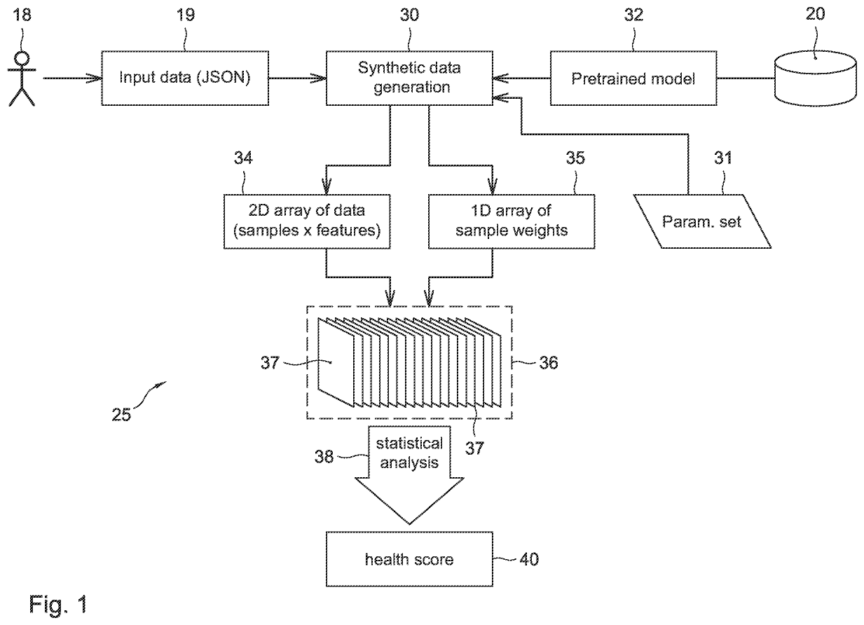

FIG. 1 schematically illustrates the process 25 in accordance with the method of the present invention. A health score 40, for example in the form of a biological age, may be determined as follows. The term ‘biological age’ relates to the term ‘physical age’ in that the ‘physical age’ indicates the real actual age of the person (i.e. the amount of time expired since the person's birth). The term ‘biological age’ in this context indicates a determined age on the basis of the actual health state of the person in consideration as compared to the mean health state of other persons having the same physical age, the latter being determinable based on statistics. For example, the occurrence of certain health conditions, habits or even environmental conditions, may positively or negatively affect the biological age in the sense that, starting from the actual physical age, these conditions may decrease the biological age when the effect of a condition is a positive factor (e.g. a healthy lifestyle e.g. by healthy nutrition, physical exercise, a weight management program) or increase the biological age when the effect of a condition is a negative factor on the overall health (e.g. smoking, or the presence of an illness). The health score 40 is not necessarily a biological age, but may also be a differently determined representation of a person's momentary health status. For example, the score may be a calculated dimensionless parameter, or may be related to a different quantifiable body parameter.

In the present embodiment, the health score 40 may be a biological age, which may for example be determined as follows. First, a large number of candidate records 37 (virtual twins) may be simulated using a first machine learning data processing model 32, based on the provided input data 19 from the user 18. The first machine learning data processing model may be a variational autoencoder (VAE) or a generative adversarial network (GAN). In a particular embodiment, the first machine learning data processing model 32 is a pre-trained Bayesian network 32. The Bayesian network model 32 may for example have been trained as described herein before (not illustrated in the figures), by performing an optimization using tabu search. The input data may include a number of different of parameter values relating to different parameters, but at least includes parameter values of the three parameters: age, gender and ethnicity. The age may be the year of age of person 18, whereas the gender may be a Boolean value indicating ‘man’ or ‘woman’, ethnicity relates to an ethnic group (e.g. black, white, Latino, Asian, multiracial). The input data contains entered data that is directly provided or made available by the user 18. The process is based on the assumption that the input data 19 as provided by the user 18 is reliable.

The entered data is to be enriched, using the pre-trained model 32, in order correct for missing parameter values in the input data 19. For example, the health score 40 ideally requires input data for a vast number of parameters, where only at least three parameter values (for age, gender and ethnicity) are provided as input data 19. The pre-trained model 32 (i.e. the first machine learning data processing model 32 referred to above) is then used to generate data for the missing parameters in step 30. The various parameter values required by the system 1 in order to perform the determination of health score 40 may be well defined, in order to allow the server 3 to exactly identify which parameters are missing from the input data 19. The exactly defined parameters together form a parameter set 31, as illustrated in FIG. 1. Step 30 thus relies on pre-trained model 32, and uses the input data 19 as well as the identified parameters in the parameter set 31, in order to identify the parameters for which entered parameter values are missing from the input data 19, and in order to generate data for the missing parameters.

This step 30 results in a dataset of candidate record 37. For example, a total of 5000 candidate records having each 29 parameter values may be formed in this manner. This would for example provide a data file 34 comprising an array of 5000 rows for each candidate record 37, and 29 columns for each parameter. The parameter values of each candidate record are either fixed to the entered parameter values of the input data 19 or simulated given conditional probabilities of the pre-trained model 32 in case these parameter values relate to parameters that are missing in the input data. Although the above identified entered parameter values for age, gender and ethnicity form an elementary set required by the system 1 (i.e. a set of minimally required parameters), the input data 19 may include further parameter values (e.g. heart rate, blood pressure, glucose levels, etc.) if these are known and made available by user 18.

Each simulated candidate record 37 also comes with a likelihood indicative of how likely it is that the candidate record 37 provides a true representation of the traits of the user 18. These likelihood values are calculated in step 30 as well, and are provided for example as a one dimensional array in data file 35 wherein each likelihood value is associated with one of the candidate records 37. This will eventually provide the set 36 of candidate records 37 as illustrated in FIG. 1, from which the health score 40 can be calculated. The various values (parameter values & likelihood values) may be structured in a different manner than the abovementioned data files 34 and 35—for example a single data file including all values may likewise be the result of step 30. This may be freely determined based on the skilled person's needs.

The set 36 of candidate records 37 thus provides a set of possible health states of the person 18, determined on the basis of the input 19, with each possible health state (i.e. candidate record 37) an associated likelihood value that determines how likely it is that the respective candidate record 37 truly applies to this particular person 18 with these input values 19. Based on these candidate records 37 and their associated likelihood values, a statistical analysis method can be applied in order to determine a health score 40 (e.g. mean health score) and, optionally but in many cases preferred, an accuracy thereof. The accuracy for example may be provided as an error value that is determined on the basis of the likelihood values of the candidate records 37 with respect to the parameter values of the estimated parameters.

In accordance with one exemplary embodiment, an individual health score may for example be calculated with each candidate record 37. For example, suppose in the above example the step of data generation 30 has resulted in 5000 candidate records 37 and associated likelihood values. Then, for each candidate record 37, an individual health status score associated with the candidate record may be determined first, such that a total of 5000 individual health status scores is obtained—the respective likelihood values of each associated candidate record 37 then apply to each of the individual health status score 40. For determining the health status score 40 of the person 18, the processor 8 is further configured for calculating a weighed mean of the individual health status scores weighed based on the associated likelihood values of each candidate record 37.

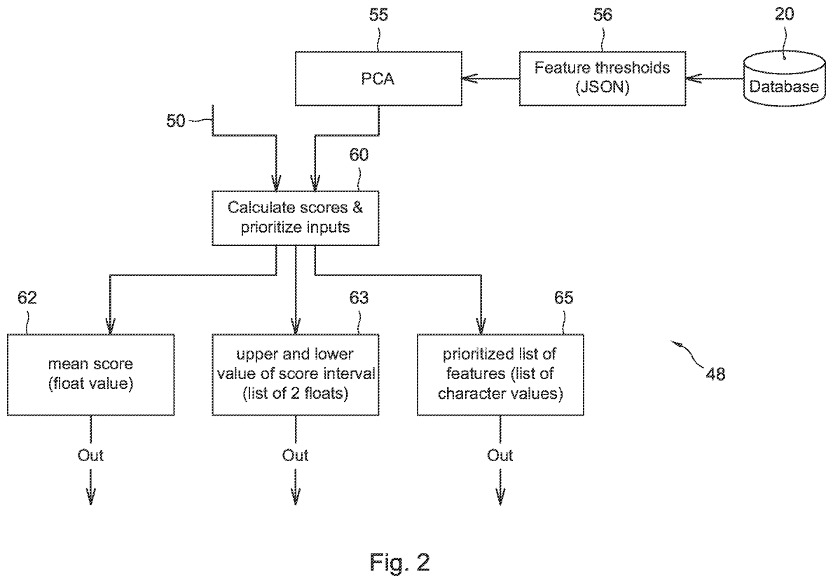

An example process 48 for determining an overall health score 40 is illustrated in FIG. 2. In these or further embodiments, to calculate the individual health scores 62 in step 60, the candidate records 37 of the set 36 are provided as input 50 to a second machine learning data processing model 55, that enables to process the large amounts of parametric data and uncertainties. For example, use may be made of a principle component analysis model 55 that may be trained on the basis of statistical data, such as data from database 20. Alternative models that may be applied as second machine learning data processing model may include an independent component analysis model (ICA), or a multidimensional scaling model (MDS), both providing similar or acceptable results. These latter two are not further explained here, but do provide good alternatives for implementation.

For training the PCA model 55, data from this database 20 may be transformed on the basis of feature thresholds 56. For example, the data is transformed based of clinical thresholds 56. For parameters expressed as continuous variables, such a threshold is subtracted from absolute values and negative values are subsequently set to zero (=0). Thus, only those parameters are included which have a parameter value above the threshold for that parameter. Thereafter, the data may be scaled or normalized, and a principal component analysis is performed to extract the scores and loadings from the first principal component.

In step 60, the trained PCA model 55 is applied in order to perform principal component analysis on the candidate records 37 in the set 36. This yields at the output thereof a collection of individual health scores 62, wherein each individual health score is associated with a candidate record 37. For these individual health scores 62, the overall health score 40 may be determined by calculating the average thereof. More preferred though, the overall health score 40 may be determined by calculating the weighted average of the individual health scores 62, wherein the weighing values are based on or equal to the likelihood values associated with each candidate record 37 and corresponding individual health score 62 thereof.

Furthermore, together the individual health scores 62 of all candidate records 37 will span a certain interval or range. The accuracy of the overall health value 40 may be represented by an error value or error margin 41. In a basic embodiment, this error margin 41 of overall health value 40 may be based on (or even provided by) this range. Alternatively, a better estimate of the error margin 41 may be determined by using the likelihood values of each candidate record 37 for calculating an upper and lower value of the score interval of the overall health score 40. This is provided at the output 63 of step 60.

Furthermore, to perform the above analysis to provide the overall health score 40 and optionally the error margin 41 thereof, the features in the candidate records 37 are prioritized. In FIG. 2, the output 65 provides the list 42 of prioritized features. As may be appreciated, this list 42 has significance in understanding how the health score 40 has to be interpreted. The priorities provided in the list 42 indicate which parameter values were of main importance in the determination of the health score 40. These priorities—like the error margin 41 and the health score 40 itself—has been constructed taking into account the uncertainties provided by the likelihood values of each candidate record 37. This is because for some candidate records 37, the hypotheses provided by the simulated parameter values may strongly influence the calculated health score 40 for that particular candidate record 37, however it may be insignificant in the end result (i.e. the overall health score 40) simply because the respective candidate record 37 has a very low likelihood. For other candidate records 37 which have a rather high likelihood value associated therewith, other parameter values with a lower priority may still have a stronger effect on the overall health score 40 due to the high likelihood value of the record 37. Therefore, clearly, the priorities will be influenced by these likelihood values, and result in a unique priorities list 42 associated with the particular health score 40, which is indicative of which conditions were considered of most importance for this person 18.

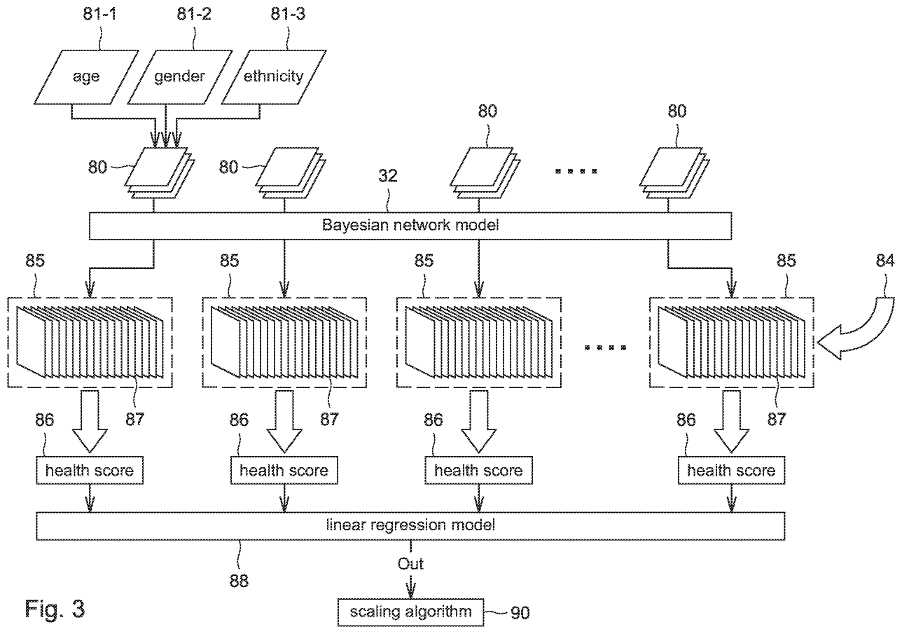

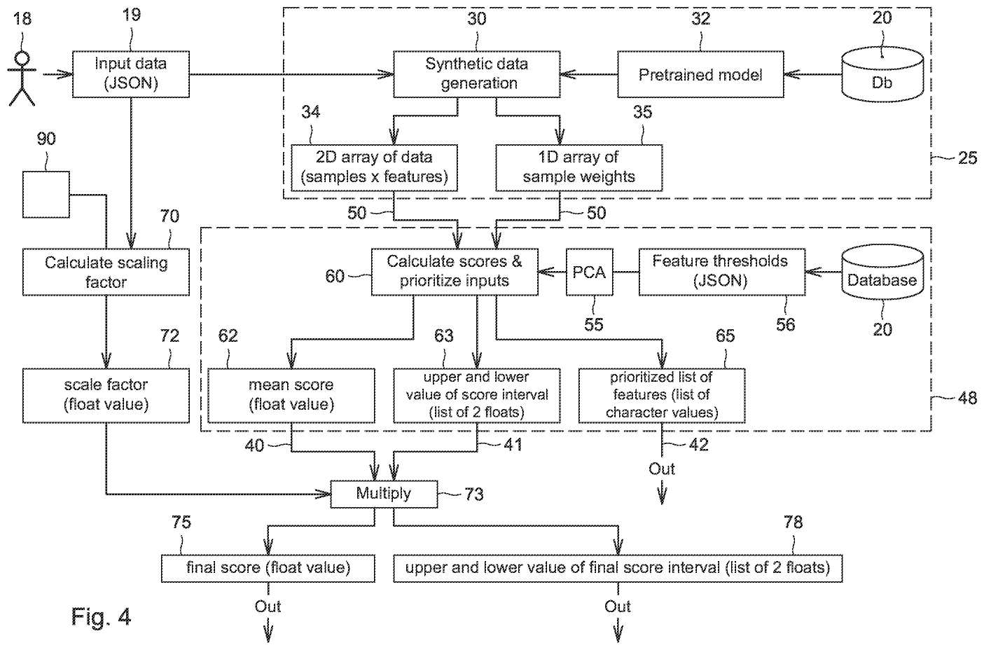

FIG. 4 provides an overview of an embodiment for calculating an overall health score 40, in accordance with an embodiment, and including the processes 25 and 48 described above. Optionally, in step 73 the determined overall health score 40 may be scaled, e.g. by multiplying the overall health score 40 with a scaling factor 72, in order to mitigate the effects of extreme situations. In particular, when dealing with human individuals that can experience stress or discouragement, a built-in scaling factor 72 is an effective manner of making an end result more palatable and useful. Such a scaling factor 72 may be calculated in step 70 using a scaling algorithm 90 determined based on a simulated dataset 84 using the trained Bayesian Network model 32 and the PCA model 55. FIG. 3 schematically illustrates how a scaling algorithm 90 may be determined using such a simulated dataset 84. First, a very large number of potential individuals 87 is virtually created by considering unique combinations 80 of the at least three parameters 81: age 81-1, gender 81-2 and ethnicity 81-3. For example, a dataset with 3150000 virtual individuals 87 may be simulated, equally distributed over 630 unique combinations 80, which 630 unique combinations are obtained over 63 age groups (18-80 years), two gender groups (M/F), and five ethnicity groups (black, white, Latino, Asian, multiracial). The virtual individuals 87 may be obtained by submitting each of the unique combinations 80 as input to the Bayesian network model 32 and determining for each combination 80 a set 85 of five thousand virtual individuals 87 (analogous to the candidate records 37 obtained in process 25). Then, for each set 85, an auxiliary health score 86 may be determined. For example, this may be done using principal component analysis model 55 (not shown in FIG. 3). A normalization factor may optionally be calculated for each unique combination 80 and associated set 85 by dividing 1 over the 95% quantile, to ensure numbers between 0 and 1. These (normalized) auxiliary health scores 86 serve as input for fitting a linear regression model 88 with gender, ethnicity, and age as dependent variables. This will yield the scaling algorithm 90 that is used to calculate the scaling factor 72 on the basis of the input 19 for person 18 in FIG. 4.

In FIG. 4, after calculating the scaling factor 72 in step 70 based on the scaling algorithm 90, the overall health score 40 is multiplied with the scaling factor 72. Where desired, the scaling factor itself may be brought in a desired proportion by multiplying it with an additional factor to provide more control over the scaling process. In embodiments wherein the overall health score 40 is desired to be expressed as a biological age 75, the latter may be obtained by adding the scaled score from step 73 to the real physical age as received via user input 19. Furthermore, the error margin 41 will likewise be scaled in step 73 by multiplication with the scaling factor 72 in the same manner, yielding a corrected error margin 78 for the biological age 75.

In the above, the parameter set 31 may consist of a plurality of defined parameters, in the sense that it is pre-determined which parameters are desired to be predicted.

For example, the parameter set 31 may include any one or more or all of the following parameters: gender; smoking status; physical age; ethnicity; heart condition history; heart rate; body mass index; arm circumference; waist circumference; hemoglobin A1c level; (overnight) fasting glucose level; glucose level at predetermined time after start of glucose tolerance test, such as after one hour, two hours or three hours; triglyceride level; high-density-lipoprotein level; low-density-lipoprotein level; total cholesterol level; diastolic blood pressure; systolic blood pressure; whether or not hemoglobin A1c level is elevated; whether or not glucose level at start of glucose tolerance test is elevated; whether or not glucose level at predetermined time after start of glucose tolerance test is elevated, such as after one hour, two hours or three hours; whether or not low-density-lipoprotein level is elevated; whether or not triglyceride level is elevated; whether or not total cholesterol level is elevated; whether or not antidiabetic medication is used; whether or not antihypertensive medication is used; whether or not antihyperlipidemic medication is used; hypertension status; presence or absence of the metabolic syndrome; presence or absence of prediabetes; maximum oxygen uptake (i.e. VO₂ max); thigh circumference; sleep duration; daily number of steps; and any ratios between quantifiable parameters, such as body length to waist circumference ratio.

In another embodiment, the parameter set 31 may include any one or more or all of the following parameters: age, gender, education level, family health history of coronary heart disease, family health history of type 2 diabetes, smoking, sleep duration, stress at work, physical activity, coffee intake, screen time, obesity, systolic blood pressure, and high-density lipoprotein (HDL) cholesterol.

In another embodiment, the parameter set 31 may include any one or more or all of the following parameters: glucose concentration, insulin concentration, C-peptide concentration, high-density lipoprotein (HDL) cholesterol, non-esterified fatty acids (NEFA), total cholesterol, triglycerides, alanine aminotransferase (ALT), aspartate aminotransferase (ASAT), beta-hydroxybutyrate, gamma-glutamyl transferase (GGT), Interleukin 10 (Il-10), Interleukin 6 (Il-6), Interleukin 8 (Il-8), tumor necrosis factor alpha (TNF-α). These parameters are typically measurements of a blood test that are, for example, taken as part of an Oral Glucose Tolerance Test (OGTT) or mixed-meal tolerance test (MMTT). Such measurements have preferably been taken before and/or after consumption of an OGTT and/or MMTT. Preferably, multiple values of a parameter are included in the dataset, representing multiple measurements of the parameter taken over time. For example, a parameter may include measurements before consumption of the OGTT and/or MMTT (t=0), 30 minutes after consumption, 60 minutes after consumption, 120 minutes after consumption, and 240 minutes after consumption.

In another embodiment, the parameter set 31 may include any one or more or all of the following parameters: arm circumference, thigh circumference, waist circumference, body-mass index, height, and 6 minute walking test.

In respect of the abovementioned parameters, the principal component analysis model 55 may be configured for calculating a single representative value of a first principal component based on one or more of the parameters of the parameter set 31 as input. These parameters may comprise one or more of: smoking status; heart condition history; heart rate; body mass index; arm circumference; waist circumference; hemoglobin A1c level; glucose level at start of glucose tolerance test; glucose level at predetermined time after start of glucose tolerance test, such as after one hour, two hours or three hours; triglyceride level; high-density-lipoprotein level; low-density-lipoprotein level; total cholesterol level; diastolic blood pressure; and systolic blood pressure.

The present invention has been described in terms of some specific embodiments thereof. It will be appreciated that the embodiments shown in the drawings and described herein are intended for illustrated purposes only and are not by any manner or means intended to be restrictive on the invention. It is believed that the operation and construction of the present invention will be apparent from the foregoing description and drawings appended thereto. It will be clear to the skilled person that the invention is not limited to any embodiment herein described and that modifications are possible which should be considered within the scope of the appended claims. Also kinematic inversions are considered inherently disclosed and to be within the scope of the invention. Moreover, any of the components and elements of the various embodiments disclosed may be combined or may be incorporated in other embodiments where considered necessary, desired or preferred, without departing from the scope of the invention as defined in the claims.

In the claims, any reference signs shall not be construed as limiting the claim. The term ‘comprising’ and ‘including’ when used in this description or the appended claims should not be construed in an exclusive or exhaustive sense but rather in an inclusive sense. Thus the expression ‘comprising’ as used herein does not exclude the presence of other elements or steps in addition to those listed in any claim. Furthermore, the words ‘a’ and ‘an’ shall not be construed as limited to ‘only one’, but instead are used to mean ‘at least one’, and do not exclude a plurality. Features that are not specifically or explicitly described or claimed may be additionally included in the structure of the invention within its scope. Expressions such as: “means for . . . ” should be read as: “component configured for . . . ” or “member constructed to . . . ” and should be construed to include equivalents for the structures disclosed. The use of expressions like: “critical”, “preferred”, “especially preferred” etc. is not intended to limit the invention. Additions, deletions, and modifications within the purview of the skilled person may generally be made without departing from the spirit and scope of the invention, as is determined by the claims. The invention may be practiced otherwise then as specifically described herein, and is only limited by the appended claims.

1. A system for determining, based on input associated with a person, a health status score associated with the person, and wherein the input relates to parameter values of one or more parameters from a parameter set, the parameter set comprising a plurality of defined parameters, the parameters relating to traits of the person, wherein the system comprises:
an input interface configured to receive the input, wherein the input comprises input data representing entered parameter values of at least three parameters from the plurality of parameters of the parameter set;
wherein the system further comprises a processor configured for executing a first machine learning data processing model;
wherein the first machine learning data processing model is configured for generating, based on the input data, a plurality of candidate records,
wherein each candidate record comprises:
the entered parameter values of the at least three parameters of the parameter set; and
a candidate parameter value for each further parameter of the parameter set different from the at least three parameters;
such that, for each candidate record, a parameter value combination formed by the entered parameter values and the candidate parameter values forms a unique combination within the plurality of parameter value combinations of the candidate records;
wherein the first machine learning data processing model is further configured for generating, for each candidate record, a likelihood value indicative of a probability that the parameter value combination of the candidate record provides a true representation of the traits of the person; and
wherein, during the generating, the first machine learning data processing model is configured for generating, for each candidate record, the candidate value for each further parameter, based on the entered parameter values.

2. The system according to claim 1, wherein the processor is further configured for determining, using the parameter value combinations of the candidate records and the likelihood values associated with the candidate records, the health status score of the person associated with the input, wherein the health status score is based on the candidate records.

3. The system according to claim 2, wherein for determining the health status score the processor is further configured for executing a second machine learning data processing model, wherein the second machine learning data processing model is configured for determining, for each candidate record, an individual health status score associated with the candidate record; and
wherein, for determining the health status score of the person, the processor is further configured for calculating a weighed mean of the individual health status scores weighed based on the associated likelihood values of each candidate record.

4. The system according to claim 3, wherein the second machine learning data processing model comprises at least one of: model taken from the group consisting of:
a principal component analysis model,
an independent component analysis model,
a multidimensional scaling model,
a singular value decomposition, and
a non-negative matrix factorization.

5. The system according to claim 1, wherein the first machine learning data processing model comprises at least one model taken from the group consisting of:
a Bayesian Network model,
a variational autoencoder, and
a generative adversarial network; and
the at least three parameters comprise age, gender and ethnicity.

6. The system according to claim 2, wherein the processor is further configured for determining, using the parameter value combinations of the candidate records and the likelihood values associated with the candidate records, an error value associated with the health status score indicative of an accuracy of the health status score.

7. The system according to claim 1, wherein the parameter set comprises one or more parameters taken from the group consisting of:
gender; smoking status; physical age; ethnicity; heart condition history; heart rate; body mass index; arm circumference; waist circumference; hemoglobin A1c level; (overnight) fasting glucose level; glucose level at predetermined time after start of glucose tolerance test; triglyceride level; high-density-lipoprotein level; low-density-lipoprotein level; total cholesterol level; diastolic blood pressure; systolic blood pressure; whether or not hemoglobin A1c level is elevated; whether or not glucose level at start of glucose tolerance test is elevated; whether or not glucose level at predetermined time after start of glucose tolerance test is elevated; whether or not low-density-lipoprotein level is elevated; whether or not triglyceride level is elevated; whether or not total cholesterol level is elevated; whether or not antidiabetic medication is used; whether or not antihypertensive medication is used; whether or not antihyperlipidemic medication is used; hypertension status; presence or absence of the metabolic syndrome; presence or absence of prediabetes; maximal oxygen uptake; thigh circumference; sleep duration; daily number of steps; and any ratios between quantifiable parameters.

8. The system according to claim 4, wherein the principal component analysis model is configured for calculating a single representative value of a first principal component based on one or more of the parameters of the parameter set as input, and
wherein the one or more parameters comprise one or more parameters taken from the group consisting of:
smoking status; heart condition history; heart rate; body mass index; arm circumference; waist circumference; hemoglobin A1c level; glucose level at start of glucose tolerance test; glucose level at predetermined time after start of glucose tolerance test; triglyceride level; high-density-lipoprotein level; low-density-lipoprotein level; total cholesterol level; diastolic blood pressure; and systolic blood pressure.

9. The system according to claim 2, wherein the processor is further configured for scaling the health status score by multiplying the health status score with a scaling factor, wherein the scaling factor is dependent on the at least three parameters.

10. The system according to claim 9, wherein the processor is configured for obtaining an algorithm for determining the scaling factor, wherein for obtaining the algorithm the processor is configured for:
identifying a plurality of distinguished conditions, wherein each condition is represented by a unique combination of parameter values of the at least three parameters;
applying the first machine learning data processing model for generating, for each condition and based on the unique combination associated with the condition, a plurality of model candidate records;
calculating, for each condition, a modelled health status score associated with the condition; and
performing a linear regression model for obtaining the algorithm.

11. The system according to claim 2, wherein the health status core is related to at least one of the group consisting of:
a physical age; and
one or more health states.

12. The system according to claim 11, wherein the health status score is related to a physical age, and
wherein the processor is further configured for calculating a biological age by adding the health status score to the physical age.

13. A method of determining, based on input associated with a person, a health status score, wherein the health status score is related to a physical age associated with the person, and wherein the input relates to parameter values of one or more parameters from a parameter set, the parameter set comprising a plurality of defined parameters, the parameters relating to traits of the person, wherein the method comprises:
receiving the input, wherein the input comprises input data representing entered parameter values of at least three parameters from the plurality of parameters of the parameter set;
generating, by a first machine learning data processing model executed by a processor, a plurality of candidate records based on the input data,
wherein each candidate record comprises:
the entered parameter values of the at least three parameters of the parameter set; and
a candidate parameter value for each further parameter of the parameter set different from the at least three parameters; and
wherein the generating is performed such that, for each candidate record, a parameter value combination formed by the entered parameter values and the candidate parameter values forms a unique combination within the plurality of parameter value combinations of the candidate records;
wherein the method further
comprises generating for each candidate record, by the first machine learning data processing model, a likelihood value indicative of a probability that the parameter value combination of the candidate record provides a true representation of the traits of the person; and
wherein during the generating, for each candidate record, generating the candidate parameter value for each further parameter is based on the entered parameter values.

14. The method according to claim 13, further comprising determining, by the processor, using the parameter value combinations of the candidate records and the likelihood values associated with the candidate records, the health status score of the person associated with the input, wherein the health status score is based on the candidate records.

15. The method according to claim 14, wherein the determining the health status score comprises:
executing, by the processor, a second machine learning data processing model, wherein the second machine learning data processing model is configured for determining, for each candidate record, an individual health status score associated with the candidate record; and
calculating a weighed mean of the individual health status scores weighed based on the associated likelihood values of each candidate record.

16. The method according to claim 15, wherein the second machine learning data processing model is a principal component analysis model.

17. The method according to claim 13, wherein at least one of:
the first machine learning data processing model is a Bayesian Network model; or
the at least three parameters comprise age, gender and ethnicity.

18. The method according to claim 13, further comprising determining, using the parameter value combinations of the candidate records and the likelihood values associated with the candidate records, an error value associated with the health status score indicative of an accuracy of the health status score.

19. The method according to claim 13, wherein the parameter set comprises one or more parameters taken from the group consisting of:
gender; smoking status; physical age; ethnicity; heart condition history; heart rate; body mass index; arm circumference; waist circumference; hemoglobin A1c level; (overnight) fasting glucose level; glucose level at predetermined time after start of glucose tolerance test; triglyceride level; high-density-lipoprotein level; low-density-lipoprotein level; total cholesterol level; diastolic blood pressure; systolic blood pressure; whether or not hemoglobin A1c level is elevated; whether or not glucose level at start of glucose tolerance test is elevated; whether or not glucose level at predetermined time after start of glucose tolerance test is elevated; whether or not low-density-lipoprotein level is elevated; whether or not triglyceride level is elevated; whether or not total cholesterol level is elevated; whether or not antidiabetic medication is used; whether or not antihypertensive medication is used; whether or not antihyperlipidemic medication is used; hypertension status; presence or absence of the metabolic syndrome; presence or absence of prediabetes; maximal oxygen uptake; thigh circumference; sleep duration; daily number of steps; and any ratios between quantifiable parameters.

20. The method according to claim 16, further comprising determining, using the principal component analysis model, a single representative value of a first principal component based on the one or more of the parameters of the parameter set as input, wherein the one or more parameters comprise one or more parameters taken from the group consisting of:
smoking status; heart condition history; heart rate; body mass index; arm circumference; waist circumference; hemoglobin A1c level; glucose level at start of glucose tolerance test; glucose level at predetermined time after start of glucose tolerance test; triglyceride level; high-density-lipoprotein level; low-density-lipoprotein level; total cholesterol level; diastolic blood pressure; and systolic blood pressure.

21. The method according to claim 14, further comprising scaling, by the processor, the health status score by multiplying the health status score with a scaling factor, wherein the scaling factor is dependent on the at least three parameters.

22. The method according to claim 21, further comprising, for performing the step of scaling, obtaining an algorithm for determining the scaling factor, wherein the obtaining the algorithm comprises:
identifying, by the processor, a plurality of distinguished conditions, wherein each condition is represented by a unique combination of parameter values of the at least three parameters;
applying, by the processor, the first machine learning data processing model for generating, for each condition and based on the unique combination associated with the condition, a plurality of model candidate records;
calculating for each condition, by the processor, a modelled health status score associated with the condition; and
performing a step of linear regression for obtaining the algorithm.

23. The method according to claim 14, wherein
the health status core is related to at least one of the group consisting of:
a physical age; or
one or more health states.

24. The method according to claim 23, wherein the health status score is related to a physical age, and
wherein the processor is further configured for calculating a biological age by adding the health status score to the physical age.

25. The method according to claim 13, further comprising training of the first machine learning data processing method, prior to the determining of the health status score, wherein the training includes:
obtaining, from a database, health statistics data, wherein the health statistics data comprises health parameter statistics for a population of persons;
performing, based on the health statistics data, an iterative optimization algorithm to identify one or more conditional dependencies between a plurality of health parameters comprised by the health statistics data, wherein the one or more conditional dependencies quantify whether and to which degree any health parameter of the plurality of health parameters is dependent on any other health parameter of the plurality of health parameters; and
terminating the iterative optimization algorithm upon identifying a stable set of conditional dependencies, wherein the set is determined as stable if upon any further iteration a change in any of the conditional dependencies is smaller than a predetermined threshold.

26. The method according to claim 25, further comprising:
obtaining a training data representing training parameter values of the at least three parameters from the plurality of parameters of the parameter set;
generating, by the first machine learning data processing model, for each further parameter of the parameter set different from the at least three parameters, a generated parameter value;
generating, by the first machine learning data processing model, a likelihood value indicative of a probability that a training combination of the training parameter values and the generated parameter values provides a true representation of the traits of the person;
comparing the likelihood value with the health parameter statistics for verifying a correctness of the likelihood value; and
modifying, dependent on the step of comparing, at least one of the one or more conditional dependencies and perform the iterative optimization algorithm.

27. The method according to claim 25, wherein the iterative optimization algorithm is a tabu search algorithm.
